(19) United States
(12) Patent Application Publication
(10) Pub. No.: US 2004/0171087 A1
(43) Pub. Date: Sep. 2, 2004

(54) Quantitative single-step immunoassay in lyophilized form

(76) Inventors: Irene Rech-Weichselbraun, Laxenburg (AT); Michael Schaude, Vienna (AT)

Correspondence Address: Robert J Schneider, Chapman & Cutler, 111 West Monroe Street, Chicago, IL, 60603, US

(21) Appl. No.: 10/476,993

(22) PCT Filed: Apr. 26, 2002
(86) PCT No.: PCT/AT02/00112

(30) Foreign Application Priority Data
May 10, 2001 (AT) GM 370/2001

(51) Int. Cl. G01N 33/53; G01N 33/537; G01N 33/543

(52) U.S. Cl. 435/7.5; 435/7.92

(57) ABSTRACT

An immunoassay kit for the qualitative and quantitative determination of a sample by the aid of specific binding partners such as, e.g., antibodies, antigens, receptors and ligands, includes a carrier or microtiter plate having a plurality of wells and precoated with a primary binding partner, which binding partner is present in fixed form as a lyophilisate. Part of the wells of the microtiter plate additionally contain a reference standard series of the sample to be determined with incremental dilutions in lyophilized form.

The invention relates to an immunoassay kit for the qualitative and quantitative determination of a sample by the aid of specific binding partners such as, e.g., antibodies, antigens, receptors and ligands, including a carrier or microtiter plate having a plurality of wells and precoated with a primary binding partner, said binding partner being present in fixed form as a lyophilisate.

Immunoassays, enzyme-coupled immunoassays, fluorescence-labeled immunoassays, luminescence-labeled immunoassays or immunoassays labeled with any other marker are used in routine diagnostics and research diagnostics to detect and quantify proteins, for instance antigens, ligands, receptors, antibodies, as well as chemical compounds. According to the prior art, one of the binding partners in such a test kit is bound to a solid carrier, for instance a polystyrene plate, while the other reagents required to perform the test are added as separate components of the test kit. These reagents usually comprise the protein to be detected, in a quantified form as a concentrate or lyophilisate (reference standard), a standard medium and a sample dilution medium, the second specific binding partner in coupled form (conjugate), as a concentrate or working solution, if necessary a secondary detection system as well as, in the event of an enzyme label, the substrate reagent required for the detection. A stop solution is added to terminate the enzymatic reaction, and an assay buffer usually present as a concentrate is available to prepare the working solutions for the second specific binding partner and the secondary reagent. Since washing steps are required between the working steps, a conventional immunoassay kit contains a washing buffer, usually in concentrated form.

Commercially available ELISA kits offer validated test systems to the user. The products contain all the reagents necessary to do the assay, at optimized concentrations and quantities, as well as a detailed protocol on how to carry out the assay. The individual reaction steps, as a rule, are carried out consecutively. The joint incubation of analytes (standard, sample) and the secondary antibody with the solid phase (cocktail) is feasible for a number of assay systems.

A typical example of an immunoassay is the sandwich ELISA (enzyme-linked immunosorbent assay).

Typically, such an ELISA kit comprises the following components:

a microtiter plate with 96 wells, precoated with the primary antibody, blocked, fixed;

a reference material: analyte (standard) at a defined concentration to establish standard series;

an enzyme- (horse radish peroxidase) or biotin-coupled secondary antibody;

if required, a streptavidin enzyme (horse radish per-oxidase)

a substrate solution (tetramethylene benzidine);

a washing buffer: saline solution to wash the plates prior to sample incubation and between reaction steps;

an assay buffer: saline solution to dilute the secondary antibody and streptavidin enzyme concentrate (preparation of working solutions);

a sample dilution medium: specific liquid medium to dilute reference material as well as unknown samples;

a stop solution to terminate the enzymatic reaction.

To perform the assay, the following working steps will have to be carried out by the user:

preparing working solutions;

establishing standard series by diluting reference material;

washing microtiter plate;

providing sample dilution medium;

applying individual reference dilutions (standard series);

applying unknown samples;

adding enzyme- or biotin-coupled secondary antibody;

washing microtiter plate;

optionally adding streptavidin enzyme;

washing microtiter plate;

adding substrate;

adding stop solution;

measuring optical density.

The invention aims to facilitate the manipulation of such assay kits and to enable the realization of a quantitative assessment in addition to a qualitative identification. In addition to reducing the work and time involved in the implementation of the assay, the invention, furthermore, aims to reduce the probability of errors for the user by minimizing the operating steps required. Finally, it is the aim of the present invention to lower packaging volumes and packaging costs as well as shipping charges by clearly reducing the weight and volume of the kits, and to facilitate logistics by providing standardized base kits with extended product storage lives.

To solve this object, the immunoassay kit according to the invention essentially consists in that part of the wells of the microtiter plate additionally comprise a reference standard series of the sample to be determined, with incremental dilutions in lyophilized form. Due to the fact that already the carrier or microtiter plate additionally comprises a reference standard series of the sample to be determined, also a quantitative estimation and quantitative determination by interpolation between defined test results in the region between incrementally consecutive standards have become feasible in addition to a purely qualitative analysis, with the cumbersome steps of preparing a working solution of the reference material and the respective dilution series during laboratory analyses being obviated. Such an immunoassay kit design which does not require the preparation of a working solution of the reference material and the introduction of the standard dilution medium into the microtiter wells provided for the standard series allows for a substantial rationalization and simplification of the method and, in particular, the provision of a precisely reproducible standard series, thus substantially reducing the probability of errors.

In a particularly advantageous manner, the prefabrication of the immunoassay kit can be further accelerated and the steps required for the determination of the sample can be further reduced. To this end, the configuration advantageously is devised such that the wells of the microtiter plate additionally contain an enzyme- or biotin-coupled conjugate of a secondary binding partner, particularly a secondary binding antibody, and in the event of said biotin-coupled conjugate, an enzyme in lyophilized form. In addition to the standard series used in defined protein concentrations to quantify the protein to be detected, also the secondary specific binding partner coupled with one of the above-mentioned labels (conjugate in titrated concentration as well as optionally secondary reagent) is, thus, introduced for detection already in lyophilized form in solid phase, together with the primary binding partner solidly bound to phases, the provision of these components in lyophilized form, i.e., in dehydrated form at low temperatures of about −30° C., freezing the reaction kinetics to such an extent that no prereactions of the reference material need be feared. Since highly diluted working solutions as well as sparingly stable solutions are not kept in stock but rather prepared on the carrier only immediately before the beginning of the test by solvent addition or rehydration, further possible error sources are excluded.

In order to further enhance, and further reduce the number of, working steps required for the determination, the configuration is devised such that the wells of the microtiter plate comprise a sample dilution medium in lyophilized form. The specific sample dilution medium can be introduced into the respective wells of the microtiter plate in the required amount at a temperature of, for instance, 20° C., while the secondary antibody-enzyme conjugate, or mixture of secondary antibody and biotin conjugate, can be introduced at the same temperature into all of the wells of the microtiter plate, whereupon the thus charged microtiter plate is subjected to lyophilization by a freeze-drying process in a freeze-drying apparatus precooled to, for instance, −30° C.

In order to complete the test kit, such a preconfectioned carrier or preconfectioned microtiter plate requires but a small number of additional components, the test kit, besides the precoated microtiter plate, advantageously comprising only a washing buffer, a substrate solution and a stop solution. For the determination of a sample it is, therefore, merely required to rehydrate the microtiter plate by adding defined volumes of distilled water to the wells of the standard series and optionally to the blanks and the sample wells, whereupon the unknown sample is charged and incubated. After having washed the microtiter plate and applied the substrate in all of the microtiter wells used for the assay, the stop solution is added after a predetermined period of time, and the test evaluation can be started immediately.

The configuration in this respect advantageously is devised such that streptavidin-horse radish peroxidase (HRP) is used as a secondary reagent in the event of a biotin-coupled secondary antibody conjugate.

In the main, the configuration according to the invention reduces the implementation of the assay for the user to the addition of an unknown sample as well as an enzymatic reaction, all other steps having previously been carried by the manufacturer of the ELISA kit. Thus, the microtiter plate is coated with the specific primary antibody, blocked and fixed by the manufacturer, whereupon the plate is cooled, for instance to −20° C., and the above-described additional steps of introducing the standard series of the sample dilution agent as well as the secondary antibody-enzyme conjugate, or mixture of secondary antibody-biotin conjugate and streptavidin enzyme, are carried out by the manufacturer in the defined manner. The implementation of the assay is, thus, reduced to adding the sample to be analyzed and evaluating the assay on the basis of the coloration of the substrate.

In a particularly advantageous manner, the immunoassay kit according to the invention is designed such that the test kit comprises peroxidase stabilizers and/or general lyoprotectants such as proteins (e.g. albumin (e.g. BSA) protein hydrolysates (peptones, gelatin, . . . ) casein), polymers (e.g. dextran, PVA, PVP), sugars (e.g. sucrose, trehalose, lactose, xylite, sorbitol, mannitol, maltose, glucose, inositol), bacteriostatic agents (e.g. thimerosal, proclin), phenolic substances and anilins, also including substituents (small alkyl residues or Cl, Br, . . . ) (e. g. o-methoxyphenol, o-methylphenol, p-methylphenol, o-aminophenol, o-hydroxybenzoic acid, (o, m or p )-hydroxybenzyl alcohol, aniline, p-aminobenzoic acid, p-methoxy-aniline, benzyl alcohol, benzoic acid, p-nitrophenol, benzylamine, 1-phenyl-1,2-ethanediol, trans-1,2-cyclohexanediol, cis-1,2-cyclohexane dicarbonic acid, cyclohexylamine); hydrophobic compounds and solvents (e.g. DMF, ethylene glycol, DMSO); detergents (e.g. Tween-20); aryl boric acid compounds (e.g. phenyl boric acid, 4-bromophenyl boric acid, 3-acetamidophenyl boric acid, 1-naphtyl boric acid); substrate analogs (e.g. TMB, luminol); polyhydroxy compounds (e.g. polyols, poly (ethylene glycol), glycerol); ectoins (e.g. (S)-2-methyl-1,4,5,6-tetrahydropyrimidine-4-carboxylic acid [THP(B)], (S,S)- β2-methyl-5-hydroxy-1,2,4,5,6-tetra-hydropyrimidine-4-carboxylic acid, [THP(A)]); ions and/or polyvalent ions (e.g. metal ions (Al, Zn, Mg, Fe, Cu, . . . )); complexing agents (e.g. EDTA); amino acids (e.g. glycine, proline, 4-hydroxyproline, serine, glutamate, alanine, lysine, sarcosine, y-aminobutyric acid, phenylalanine) and/or substances like TRIS, salts, amines, Na-cholates, sucrose monolaurate, 2-O-β-mannosyl glycerate.

Bearing in mind the particularly simple mode of procedure, the present immunoassay kit is suitable for a plurality of application options. In a particularly advantageous manner, the use of an immunoassay kit of this type is for the application in research diagnostics and in vitro diagnostics, for the application in germ serology such as, for instance, the detection of infections by HIV, HepV, EBV, CMV etc., for the application in tumor diagnostics such as, for instance, the detection of tumor markers or tumor-associated proteins, vascularization markers, metastazation markers etc., for the application in allergology such as, for instance, the detection of animal allergens, plant pollens, food components etc., for the application in autoimmune diagnostics such as, for instance, the detection of auto-antigens in the fields of ANA/ENA, diabetes mellitus/IDDM, thyroid antigens, autoimmune hepatitis, APS etc., for the detection of immunologic disorders caused by inflammatory processes or infections such as, for instance, the detection of cytokines, adhesion molecules, chemokines etc., for the detection of changes in the hormone balance such as, for instance, ovulation tests, pregnancy tests etc., for the application in therapy monitoring such as, for instance, the detection of therapeutic antibodies and antigens, organic and inorganic compounds, for the detection of cell death by apoptosis or necrosis and/or for the detection of genetic changes, genetic diseases.

In the following, the invention will be explained in more detail by way of comparison to the prior art, the prior art being referred to as standard kit.

1. Components of ELISA Kits:

a) Directly Enzyme-Coupled Conjugate

| | | | Single-step Kit according to |
|---|---|---|---|
| | Standard Kit | | Invention |
| 1 | Antibody-coated | 1 | Antibody-coated microtiter plate |
| | microtiter plate | | including standard series, |
| | | | sample dilution medium, |
| | | | conjugate antibody |
| 2 | Standard material | | |
| 3 | Assay buffer | | |
| 4 | Conjugate antibody | | |
| 5 | Sample dilution medium | | |
| 6 | Washing buffer | 2 | Washing buffer |
| 7 | Substrate solution | 3 | Substrate solution |
| 8 | Stop solution | 4 | Stop solution |

Second Antibody Coupled to Biotin, Secondary Reagent Streptavidin-Horse Radish Peroxidase (HRP) or Similar.

| | | | Single-step Kit according |
|---|---|---|---|
| | Standard Kit | | to Invention |
| 1 | Antibody-coated | 1 | Antibody-coated microtiter |
| | microtiter plate | | plate including standard |
| | | | series, sample dilution medium, |
| | | | biotinylated antibody, |
| | | | streptavidin-HRP or similar |
| 2 | Standard material | | |
| 3 | Assay buffer | | |
| 4 | Biotinylated antibody | | |
| 5 | Streptavidin-HRP or the | | |
| | similar | | |
| 6 | Sample dilution medium | | |
| 7 | Washing buffer | 2 | Washing buffer |
| 8 | Substrate solution | 3 | Substrate solution |
| 9 | Stop solution | 4 | Stop solution |

Operating Steps for Implementation of Assay:

Directly Enzyme-Coupled Conjugate

| | | | Single-step Kit according |
|---|---|---|---|
| | Standard Kit | | to Invention |
| 1 | Washing antibody-coated | | |
| | microtiter plate | | |
| 2 | Preparing working | | |
| | solution of reference | | |
| | material (standard) | | |
| 3 | Applying standard | | |
| | dilution medium into | | |
| | microtiter wells | | |
| | provided for standard | | |
| | series | | |
| 4 | External or internal | | |
| | (within plate) dilution | | |
| | of working solution of | | |
| | standard in defined | | |
| | concentrations (standard | | |
| | series) | | |
| 4a | Optionally introducing | | |
| | standard dilutions into | | |
| | respective microtiter | | |
| | wells | | |
| 5 | Applying sample dilution | | |
| | medium as zero value | | |
| | (blank) into respective | | |
| | microtiter wells | | |
| 6 | Introducing required | 1 | Rehydrating microtiter plate |
| | volume of sample | | (adding defined volumes of |
| | dilution medium into | | distilled water into wells of |
| | respective microtiter | | standard series, blanc and |
| | wells | | sample wells) |
| 7 | Applying unknown samples | 2 | Applying unknown samples |
| 8 | Preparing working | | |
| | solution of HRP | | |
| | conjugate or similar | | |
| 9 | Applying HRP conjugate | | |
| | (or similar) into all | | |
| | microtiter wells used | | |
| | for the assay | | |
| 10 | Incubation | 3 | Incubation |
| 11 | Washing microtiter plate | 4 | Washing microtiter plate |
| 12 | Applying substrate in | | Applying substrate in all |
| | all microtiter wells | | microtiter wells used for the |
| | used for the assay | | assay |
| 13 | Adding stop solution | 5 | Adding stop solution |
| 14 | Evaluating assay | 6 | Evaluating assay |

b) Second Antibody Coupled to Biotin, Secondary Reagent Streptavidin-Horse Radish Peroxidase (HRP) or Similar.

| | | | Single-step Kit according |
|---|---|---|---|
| | Standard Kit | | to Invention |
| 1 | Washing antibody-coated | | |
| | microtiter plate | | |
| 2 | Preparing working | | |
| | solution of reference | | |
| | material (standard) | | |
| 3 | Applying standard | | |
| | dilution medium into | | |
| | microtiter wells | | |
| | provided for standard | | |
| | series | | |
| 4 | External or internal | | |
| | (within plate) dilution | | |
| | of working solution of | | |
| | standard in defined | | |
| | concentrations (standard | | |
| | series) | | |
| 4a | Optionally introducing | | |
| | standard dilutions into | | |
| | respective microtiter | | |
| | wells | | |
| 5 | Applying sample dilution | | |
| | medium as zero value | | |
| | (blank) into respective | | |
| | microtiter wells | | |
| 6 | Introducing required | 1 | Rehydrating microtiter plate |
| | volume of sample | | (adding defined volumes of |
| | dilution medium into | | distilled water into wells of |
| | respective microtiter | | standard series, blanc and |
| | wells | | sample wells) |
| 7 | Applying unknown samples | 2 | Applying unknown samples |
| 8 | Preparing working | | |
| | solution of biotinylated | | |
| | antibody | | |
| 9 | Applying biotin | | |
| | conjugate into all | | |
| | microtiter wells used | | |
| | for the assay | | |
| 10 | Incubation | | |
| 11 | Washing microtiter plate | | |
| 12 | Preparing streptavidin- | | |
| | HRP or similar working | | |
| | solution | | |
| 13 | Applying streptavidin- | | |
| | HRP or similar solution | | |
| | into all microtiter | | |
| | wells used for the assay | | |
| 14 | Incubation | 3 | Incubation |
| 15 | Washing microtiter plate | 4 | Washing microtiter plate |
| 16 | Applying substrate in | | Applying substrate in all |
| | all microtiter wells | | microtiter wells used for the |
| | used for the assay | | assay |
| 17 | Adding stop solution | 5 | Adding stop solution |
| 18 | Evaluating assay | 6 | Evaluating assay |

The invention may be applied for the detection of antigens by the aid of an antibody couple (sandwich ELISA). Likewise, the detection of antibodies by the respective antigen is feasible. The detection of receptors or ligands by the respective binding partner is another option to apply the present invention, a huge number of test formats being thus available. The assay may be used, in particular, for the detection of proteins, steroids, chemical compounds, drugs, nucleic acids and similar substances.

The detection of the reaction complex may be effected by the aid of a directly coupled enzyme (for instance horse radish peroxidase, alkaline phosphatase and the like), through an enzymatic reaction occurring in a secondary step (biotin-streptavidin-HRP, enzyme-coupled antibody against detection antibody and the like). Any other label suitable for use in immunoassays, such as fluorochromes, chemiluminescence labels, radioactive labels and the like, may be used as well.

The carrier used to immobilize the primary binding partner is preferably a polystyrene 96-well microtiter plate, yet any other carrier suitable for use in immunoassays may likewise be employed to carry out the present invention.

All of the samples used for measurement may be comprised of liquid samples containing the analyte, and more specifically, body liquids such as sera, plasma preparations, local body liquids, whole blood and the like, as well as cell culture supernatants, buffered analyte solutions and the like.

In the following, the invention will be explained in more detail by way of specific application examples.

### APPLICATION EXAMPLE 1

### Sandwich ELISA for the Detection of Human ICAM-1

A) Manufacture of the Plates in Connection with Sample Dilution Medium, Standard Series, HRP Conjugate

Step 1:

Coating:

The 96-well microtiter plates are coated according to the prior art with 2.5 μg/ml anti-ICAM-1 in PBS buffer (100 μl per well, incubation over night at 4° C.).

Step 2:

Blocking:

The coating solution is sucked off and the plate is washed twice with washing buffer (PBS/Tween). In order to saturate the polystyrene surface (prevention of unspecific bonds), the plate is blocked with 300 μl per well of PBS/2% BSA (2 h, RT).

Step 3:

Fixation:

The blocking solution is sucked off, the plate is washed twice, 150 μl PBS/15% sucrose are added per well. After a one-hour incubation at RT, the fixing solution is decanted. The plate is dried in the circulating drier at 280° C. for about 20 hours. The plate is frozen to −20° C.

Step 4:

Introduction of Sample Dilution Medium:

The coated plate is used in the frozen state. The sample dilution medium is cooled to 2° C. In order to establish the standard series, 100 μl sample dilution medium is introduced into each well in the first two rows of the microtiter plate. 100 μl sample dilution medium is added to the respective wells for blank determination. 90 μl sample dilution medium is added to each respective well in order to determine the unknown samples.

Step 5:

Establishment of Standard Series:

The working solution (20 ng/ml) of the reference material (recombinant human ICAM-1) (2° C.) is introduced into the two uppermost wells on the left side of the microtiter plate (100 μl/well) in double determination. A standard series is prepared (10 ng/ml; 5 ng/ml; 2.5 ng/ml; 1.25 ng/ml; 0.63 ng/ml) by a serial 1:2 dilution in the plate.

Step 6:

Introduction of HRP Conjugate:

The working solution of the HRP conjugate (anti-ICAM-1-HRP) is cooled to 2° C. and rapidly introduced into all wells of the microtiter plate (50 μl/well).

Step 7:

Lyophilization:

The microtiter plate is covered by a foil immediately after charging with all of the components and introduced into the freeze-drying apparatus precooled to −30° C. Lyophilization is effected at −30° C. for about 20 hours. The dried plate is welded into an aluminum bag immediately upon removal from the freeze-drying apparatus together with a desiccant.

B) Assay Implementation:

The plate is removed from the aluminum bag.

The standard series as well as the blanks are rehydrated by the addition of 150 μl distilled water per well, the sample wells are rehydrated by the addition of 140 μl H₂O. 10 μl of the unknown sample is each applied into the sample wells. The plate is covered by a foil and incubated for 1 hour at RT. The plate is washed three times, 100 μl substrate solution is added into each well of the plate. The enzymatic reaction is stopped after 15 minutes by the addition of the stop solution (100 μl) and the color intensity in the individual wells is photometrically evaluated.

### APPLICATION EXAMPLE 2

### Sandwich ELISA for the Detection of Human Interleukin-10 (IL-10)

A) Manufacture of the Plates in Connection with Sample Dilution Medium, Standard Series, Biotin Conjugate, Streptavidin-HRP

Step 1:

Coating:

The 96-well microtiter plates are coated according to the prior art with 5 μg/ml anti-IL-10 in PBS buffer (100 μl per well, incubation over night at 4° C.).

Step 2:

Blocking:

The coating solution is sucked off and the plate is washed once with washing buffer (PBS/Tween). In order to saturate the polystyrene surface (prevention of unspecific bonds), the plate is blocked with 300 μl per well of PBS/2% BSA (2 h, RT).

Step 3:

Fixation:

The blocking solution is sucked off, the plate is washed twice, 150 μl PBS/15% sucrose is added per well. After a one-hour incubation at RT, the fixing solution is decanted. The plate is dried in the circulating drier at 28° C. for about 20 hours. The plate is frozen to −20° C.

Step 4:

Introduction of Sample Dilution Medium:

The coated plate is used in the frozen state. The sample dilution medium is cooled to 2° C. In order to establish the standard series, 100 μl sample dilution medium is introduced into each well in the first two rows of the microtiter plate. 100 μl sample dilution medium is added into the respective wells for blank determination. 50 μl sample dilution medium is added into each well in order to determine the unknown samples.

Step 5:

Establishment of Standard Series:

The working solution (400 pg/ml) of the reference material (recombinant human IL-10) (2° C.) is introduced into the two uppermost wells on the left side of the microtiter plate (100 μl/well) in double determination. A standard series is prepared by a serial 1:2 dilution (200-3.1 pg/ml) in the plate.

Step 6:

Introduction of Biotin Conjugate and Streptavidin-HRP:

The working solution of the mixture of biotin conjugate (anti-IL-10-BT) and streptavidin-HRP is cooled to 2° C. and rapidly introduced into all wells of the microtiter plate (50 μl/well).

Step 7:

Lyophilization:

The microtiter plate is covered by a foil immediately after charging with all of the components and introduced into the freeze-drying apparatus precooled to −30° C. Lyophilization is effected at −30° C. for about 20 hours. The dried plate is welded into an aluminum bag immediately upon removal from the freeze-drying apparatus together with a desiccant.

B) Assay Implementation:

The plate is removed from the aluminum bag.

The standard series as well as the blanks are rehydrated by the addition of 150 μl distilled water per well, the sample wells are rehydrated by the addition of 100 μl H₂O. 50 μl of the unknown sample is each applied into the sample wells. The plate is covered by a foil and incubated for 3 hours at RT. The plate is washed three times, 100 μl substrate solution is added into each well of the plate. The enzymatic reaction is stopped after 15 minutes by the addition of the stop solution (100 μl) and the color intensity in the individual wells is photometrically evaluated.

### APPLICATION EXAMPLE 3

### Inverse Sandwich ELISA for the Detection of Human Antibodies Against Interferon Alpha (IFN_)

A) Manufacture of the Plates in Connection with Sample Dilution Medium, Standard Series, HRP Conjugate

Step 1:

Coating:

The 96-well microtiter plates are coated according to the prior art with 10 μg/ml streptavidin in PBS buffer (100 μl per well, incubation over night at 4° C.).

Step 2:

Specific Coating/Blocking:

The streptavidin coating solution is sucked off and the plate is washed once with washing buffer (PBS/Tween). In order to specifically coat the plate and saturate the polystyrene surface (prevention of unspecific bonds), the plate is coated, and blocked, respectively, with 300 μl per well of IFNα-biotin conjugate, 1 μg/ml in PBS/2% BSA, (2 h, 37° C.).

Step 3:

Fixation:

The coating/blocking solution is sucked off, the plate is washed twice, 150 μl PBS/15% sucrose is added per well. After a one-hour incubation at RT, the fixing solution is decanted. The plate is dried in the circulating drier at 28° C. for about 20 hours. The plate is frozen to −20° C.

Step 4:

Introduction of Sample Dilution Medium:

The coated plate is used in the frozen state. The sample dilution medium is cooled to 2° C. In order to establish the standard series, 100 μl sample dilution medium is introduced into each well in the first two rows of the microtiter plate. 100 μl sample dilution medium is added into the respective wells for blank determination. 75 μl sample dilution medium is added into each well in order to determine the unknown samples.

Step 5:

Establishment of Standard Series:

The working solution (200 ng/ml) of the reference material (anti-human IFN_antibody) (2° C.) is introduced into the two uppermost wells on the left side of the microtiter plate (100 μl/well) in double determination. A standard series is prepared by a serial 1:2 dilution (100-1.6 ng/ml).

Step 6:

Introduction of HRP Conjugate:

The working solution of the HRP-coupled IFN_protein is cooled to 2° C. and rapidly introduced into all wells of the microtiter plate (50 μl/well).

Step 7:

Lyophilization:

The microtiter plate is covered by a foil immediately after charging with all of the components and introduced into the freeze-drying apparatus precooled to −30° C. Lyophilization is effected at −30° C. for about 20 hours. The dried plate is welded into an aluminum bag immediately upon removal from the freeze-drying apparatus together with a desiccant.

B) Assay Implementation:

The plate is removed from the aluminum bag.

The standard series as well as the blanks are rehydrated by the addition of 150 μl distilled water per well, the sample wells are rehydrated by the addition of 125 μl H₂O. 25 μl of the unknown sample are each applied into the sample wells. The plate is covered by a foil and incubated for 2 hours at RT. The plate is washed three times, 100 μl substrate solution is added into each well of the plate. The enzymatic reaction is stopped after 15 minutes by the addition of the stop solution (100 μl) and the color intensity in the individual wells is photometrically evaluated.

### APPLICATION EXAMPLE 4

### BioLISA for the Detection of Human Tumor Necrosis Factor Alpha (TNFα) (Receptor-Ligand Bond)

A) Manufacture of the Plates in Connection with Sample Dilution Medium, Standard Series, Biotin Conjugate, Streptavidin-HRP

Step 1:

Coating:

The 96-well microtiter plates are coated according to the prior art with 1 μg/ml recombinant TFN receptor in PBS buffer (100 μl per well, incubation over night at 4° C.).

Step 2:

Blocking:

The coating solution is sucked off and the plate is washed once with washing buffer (PBS/Tween). In order to saturate the polystyrene surface (prevention of unspecific bonds), the plate is blocked with 300 μl per well of PBS/2% BSA (2 h, RT).

Step 3:

Fixation:

The blocking solution is sucked off, the plate is washed twice, 150 μl PBS/15% sucrose are added per well. After a one-hour incubation at RT, the fixing solution is decanted. The plate is dried in the circulating drier at 28° C. for about 20 hours. The plate is frozen to −20° C.

Step 4:

Introduction of Sample Dilution Medium:

The coated plate is used in the frozen state. The sample dilution medium is cooled to 2° C. In order to establish the standard series, 100 μl sample dilution medium is introduced into each well in the first two rows of the microtiter plate. 100 μl sample dilution medium is added into the respective wells for blank determination. 50 μl sample dilution medium is added into each well in order to determine the unknown samples.

Step 5:

Establishment of Standard Series:

The working solution (2000 pg/ml) of the reference material (recombinant TFNα) (2° C.) is introduced into the two uppermost wells on the left side of the microtiter plate (100 μl/well) in double determination. A standard series is prepared by a serial 1:2 dilution (1000-16 pg/ml) in the plate.

Step 6:

Introduction of Biotin Conjugate and Streptavidin-HRP:

The working solution of the biotin conjugate (anti-TFNαBT) and streptavidin-HRP mixture is cooled to 2° C. and rapidly introduced into all wells of the microtiter plate (50 μl/well)

Step 7:

Lyophilization:

The microtiter plate is covered by a foil immediately after charging with all of the components and introduced into the freeze-drying apparatus precooled to −30° C. Lyophilization is effected at −30° C. for about 20 hours. The dried plate is welded into an aluminum bag immediately upon removal from the freeze-drying apparatus together with a desiccant.

B) Assay Implementation:

The plate is removed from the aluminum bag.

The standard series as well as the blanks are rehydrated by the addition of 150 μl distilled water per well, the sample wells are rehydrated by the addition of 100 μl H₂O. 50 μl of the unknown sample are each applied into the sample wells. The plate is covered by a foil and incubated over night at 4° C. The plate is washed three times, 100 μl substrate solution is added into each well of the plate. The enzymatic reaction is stopped after 15 minutes by the addition of the stop solution (100 μl) and the color intensity in the individual wells is photometrically evaluated.

1. An immunoassay kit for the qualitative and quantitative determination of a sample by the aid of specific binding partners such as, e.g., antibodies, antigens, receptors and ligands, including a carrier or microtiter plate having a plurality of wells and precoated with a primary binding partner, said binding partner being present in fixed form as a lyophilisate, characterized in that part of the wells of the microtiter plate additionally comprise a reference standard series of the sample to be determined with incremental dilutions in lyophilized form.

2. An immunoassay kit according to claim 1, characterized in that the wells of the microtiter plate additionally contain an enzyme- or biotin-coupled conjugate of a secondary binding partner, particularly a secondary binding antibody, and in the event of a biotin-coupled conjugate, an enzyme in lyophilized form.

3. An immunoassay kit according to claim 1 or 2, characterized in that the wells of the microtiter plate comprise a sample dilution medium in lyophilized form.

4. An immunoassay kit according to claim 1, 2 or 3, characterized in that the test kit, besides the precoated microtiter plate, comprises only a washing buffer, a substrate solution and a stop solution.

5. An immunoassay kit according to any one of claims 1 to 4, characterized in that streptavidin-horse radish peroxidase (HRP) is used as a secondary reagent in the event of a biotin-coupled secondary antibody conjugate.

6. An immunoassay kit according to any one of claims 1 to 5, characterized in that the test kit comprises peroxidase stabilizers and/or general lyoprotectants such as proteins (e.g. albumin (e.g. BSA) protein hydrolysates (peptones, gelatin, . . . ) casein) and/or polymers (e.g. dextran, PVA, PVP), sugars (e.g. sucrose, trehalose, lactose, xylite, sorbitol, mannitol, maltose, glucose, inositol) and/or bacteriostatic agents (e.g. thimerosal, proclin) and/or phenolic substances and anilins, also including substituents (small alkyl residues or Cl, Br, . . . ) (e. g. o-methoxyphenol, o-methylphenol, p-methylphenol, o-aminophenol, o-hydroxybenzoic acid, (o, m or p)-hydroxybenzyl alcohol, aniline, p-aminobenzoic acid, p-methoxy-aniline, benzyl alcohol, benzoic acid, p-nitrophenol, benzylamine, 1-phenyl-1,2-ethanediol, trans-1,2-cyclohexanediol, cis-1,2-cyclohexane dicarbonic acid, cyclohexylamine) and/or hydrophobic compounds and/or solvents (e.g. DMF, ethylene glycol, DMSO) and/or detergents (e.g. Tween-20) and/or aryl boric acid compounds (e.g. phenyl boric acid, 4-bromophenyl boric acid, 3-acetamidophenyl boric acid, 1-naphtyl boric acid) and/or substrate analogs (e.g. TMB, luminol) and/or polyhydroxy compounds (e.g. polyols, poly (ethylene glycol), glycerol) and/or ectoins (e.g. (S)-2-methyl-1,4,5,6-tetrahydropyrimidine-4-carboxylic acid [THP(B)], (S,S)-β-2-methyl-5-hydroxy-1,2,4,5,6-tetra-hydropyrimidine-4-carboxylic acid, [THP(A)]) and/or ions and/or polyvalent ions (e.g. metal ions (Al, Zn, Mg, Fe, Cu, . . . )) and/or complexing agents (e.g. EDTA) and/or amino acids (e.g. glycine, proline, 4-hydroxyproline, serine, glutamate, alanine, lysine, sarcosine, y-aminobutyric acid, phenylalanine) and/or substances like TRIS, salts, amines, Na-cholates, sucrose monolaurate and/or 2-O-β-mannosyl glycerate.

7. The use of an immunoassay kit according to any one of claims 1 to 6 for the application in research diagnostics and in vitro diagnostics, for the application in germ serology such as, for instance, the detection of infections by HIV, HepV, EBV, CMV etc., for the application in tumor diagnostics such as, for instance, the detection of tumor markers or tumor-associated proteins, vascularization markers, metastazation markers etc., for the application in allergology such as, for instance, the detection of animal allergens, plant pollens, food components etc., for the application in autoimmune diagnostics such as, for instance, the detection of auto-antigens in the fields of ANA/ENA, diabetes mellitus/IDDM, thyroid antigens, autoimmune hepatitis, APS etc., for the detection of immunologic disorders caused by inflammatory processes or infections such as, for instance, the detection of cytokines, adhesion molecules, chemokines etc., for the detection of changes in the hormone balance such as, for instance, ovulation tests, pregnancy tests etc., for the application in therapy monitoring such as, for instance, the detection of therapeutic antibodies and antigens, organic and inorganic compounds, for the detection of cell death by apoptosis or necrosis and/or for the detection of genetic changes, genetic diseases.
